(19) United States
(12) Patent Application Publication
(10) Pub. No.: US 2015/0025349 A1
(43) Pub. Date: Jan. 22, 2015

(54) PAD FOR MEASURING BLOOD LOSS

(71) Applicants: WILLIAM MARSH RICE UNIVERSITY, HOUSTON, TX (US); BAYLOR COLLEGE OF MEDICINE, HOUSTON, TX (US)

(72) Inventors: Z. Maria Oden, Houston, TX (US); Ana Fey El-Behadli Gonzalez, La Florida (VE); Lila Kerr, New Haven, CT (US); Chethan Ramprasad, Cincinnati, OH (US); Amit Suneja, Katy, TX (US); Katherine Emily Barnett, Dallas, TX (US); Rebecca Rae Richards-Kortum, Houston, TX (US); Manisha Gandhi, Houston, TX (US)

(21) Appl. No.: 14/378,241

(22) PCT Filed: Feb. 12, 2013
(86) PCT No.: PCT/US2013/025781

§ 371 Date: Aug. 12, 2014

Related U.S. Application Data
(60) Provisional application No. 61/598,232, filed on Feb. 13, 2012.

(51) Int. Cl. A61B 5/02; A61F 13/84; A61F 13/472

(52) U.S. Cl. CPC A61B 5/02042; A61F 13/472; A61F 13/84; A61F 2013/1517
USPC 600/371; 604/385.01; 604/367; 604/377; 493/374

(57) ABSTRACT

A pad is disclosed having a grid of absorbent structures (e.g., squares) formed on the pad. The pad may be used to quantify or otherwise assess blood loss by a patient. For example, blood lost by the patient and absorbed by the pad may be quantified by counting the number of saturated squares on the pad and taking into account the known absorbance of each square.

### CROSS-REFERENCE TO RELATED APPLICATIONS

This application claims priority to and the benefit of a Provisional Patent Application No. 61/598,232, entitled “PAD FOR MEASURING BLOOD LOSS” and filed Feb. 13, 2012 and PCT Application No. PCT/US2013/025781, entitled “PAD FOR MEASURING BLOOD LOSS” and filed Feb. 12, 2013, which is herein incorporated by reference in its entirety for all purposes.

### BACKGROUND

The present disclosure relates generally to the measurement of blood loss.

Post-Partum hemorrhage is the world's leading cause of maternal mortality, occurring in 13,795,000 births and remaining responsible for 140,000 maternal deaths yearly. Accurate measurement of blood loss, needed to diagnose and treat the condition, is extremely difficult. The most common method of measuring blood loss is visual estimation, but this approach has repeatedly proven to be scientifically inaccurate.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

An absorbent pad is provided that guides visual estimation of blood loss. In one implementation, the pad is divided into a grid of uniformly sized squares that each contain a fixed amount of fixed amount of super-absorbent polymer. An example of one such suitable polymer is the polymer typically employed in disposable diapers.

In one example of a use for such a pad, the pad is placed under a woman after she has delivered a baby but before she delivers the placenta. As the woman loses blood, each square of the pad will become saturated, indicated by an easily identifiable color change and the swelling or expansion of the respective square. The number of squares that are saturated may then be counted and this number may be converted into, or used to estimate, the volume of blood lost. The volume of lost blood may then be used to make an informed diagnosis.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a partial cut away view of a pad, in accordance with aspects of the present disclosure;

FIG. 2 depicts a graph of an comparative accuracy assessment for a pad, as disclosed in accordance with aspects of the present disclosure; and

FIG. 3 depicts a graph of an experimentally determined actual versus estimated blood volume using a pad, as disclosed in accordance with aspects of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, any calculations, correlations, and/or measurements discussed herein may be performed using one or more suitable computer-implemented algorithms, such as may be stored on a computer-readable memory or medium for execution by a suitable processing component in communication with the medium or memory. In addition, previously determined constants or correlations may be stored on the computer-readable memory or medium and accessed by the processing component during execution of the algorithms. The processor may also receive inputs or measurements from a measurement device or from personnel. Such inputs may be processed used as inputs to the algorithms during execution of the algorithms. Examples of devices having suitable processing components, memory components, user interface circuitry, and/or circuitry for accessing local or remote media include, but are not limited to, desktop, notebook, and tablet computers, personal digital assistants, cellular telephones, media players, and so forth.

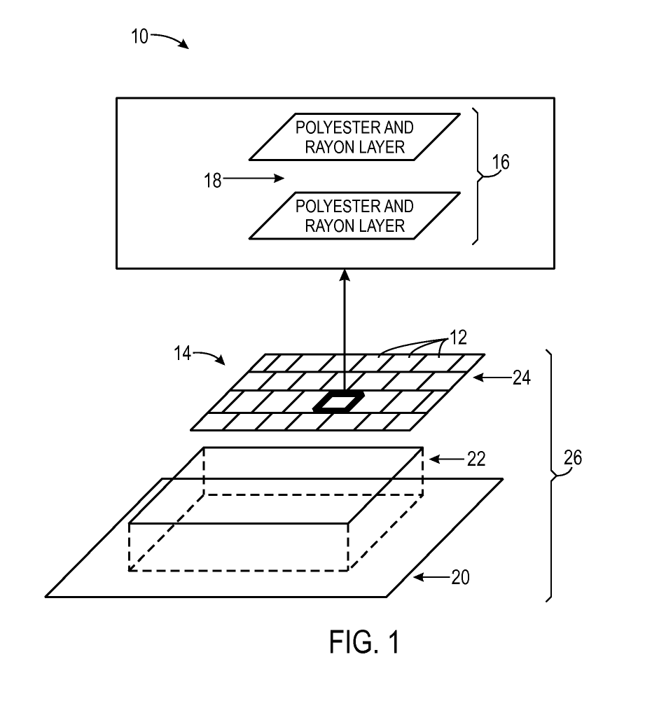

As discussed herein, a pad is provided that will absorb blood loss in a uniform pattern that allows a birth attendant to easily estimate blood loss volumes. In one embodiment, the pad is based on a rectangular absorbent pad. In one such implementation, the pad 10 has three inch squares 12 in a five-by-five grid 14, as depicted in FIG. 1. In the depicted example, each individual square 12 is composed of two layers 16 of this fabric (such as polyester and/or rayon layers) which may or may not be of different thicknesses. In other implementations, a different number of layers 16 may be present, such as four or six layers 16. In one implementation, each 3-inch square 12 on the grid 14 will contain a fixed amount of sodium polyacrylate (or a similar absorbent polymer) (e.g., powder 18) which will absorb a known amount of blood. Together the fabric layers 16 and powder 18 form an absorbent fabric layer 24. In an embodiment, absorbent fabric layer 24 is sewn or otherwise attached to a thin plastic layer 20 having a layer 22 of matted cotton (which together form a base or backing layer 26. In other embodiments, glue or adhesion may be used in place of sewing to form the squares 12 and/or to absorbent fabric layer 24 to the backing layer 26.

Immediately after a baby is delivered, the birth attendant will place the pad 10 under the woman so that the blood lost will be collected on the pad 10. As the woman loses blood, each square 12 would absorb blood until it has reached its known capacity. By counting the number of squares 12 that have become saturated, the birth attendant would be able to calculate the total blood loss. For example, if each square 12 absorbs 50 mL and the woman's blood loss saturates five squares 12, the birth attendant would know that that woman has lost 250 mL of blood. It is possible that some squares 12 would be only partially saturated, in which case they could be counted as a half-square or some other suitable fraction of a square 12. In one such embodiment, the pad 10 is capable of measuring blood loss volume with a 100 mL resolution.

With respect to the construction of the pad 10, in one embodiment, the pad 10 is constructed using fabric, super-absorbent polymer, and thread. In one such construction process, 3-inch squares 12 of fabric are cut. Two or four of these fabric squares 12 are layered (layers 16) and sewn together on three of the four sides. A fixed amount of super absorbent polymer is placed into each square 12 and the squares are sewn closed. Twenty-five such squares are sewn together to make a quilt-like grid 14. The resulting five-by-five grid 14 is sewn to a matted cotton layer 22 and plastic layer 20.

A pad 10 as described herein will allow a health care worker to measure blood loss, such as to diagnose postpartum hemorrhage. While the above-described pad 10 has been discussed in the context of assessing postpartum hemorrhage, it should be appreciated that the pad 10 may be used to assess or measure blood loss in other contexts, may be used to assess or measure the loss of fluids other than blood, and/or may be used in other contexts, medical or non-medical in nature.

With the foregoing discussion in mind, various tests were performed to determine the efficacy of a pad as described herein for measuring blood loss. In one such test, the accuracy of blood loss estimation was assessed using a pad, as discussed herein, compared to a Chux pad commonly used in a medical environment for absorbing bodily fluids and which does not include features (i.e., guides) for visual estimation of absorbed fluids.

In this test, participants estimated blood loss volumes by looking at photos, shown in an online survey, of pads as discussed herein. Participants included undergraduate students, graduate students, and healthcare professionals. Photos of known volumes were taken at the increments of 200 mL, 350 mL, 400 mL, 500 mL, 650 mL, of blood. Participants were instructed to estimate the blood loss shown in the picture and the estimates were collected. In addition, participants also estimated blood loss volumes shown on a Chux pad, an absorbent pad without any guided visual estimation, as a control. Photos were taken of known volumes of blood at increments of 200 mL, 350 mL, 400 mL, 500 mL, and 650 mL of blood. Data was also collected on the subjective assessment of the participants with respect to the ease of use of both the pad as disclosed herein and the Chux pads for estimating fluid loss.

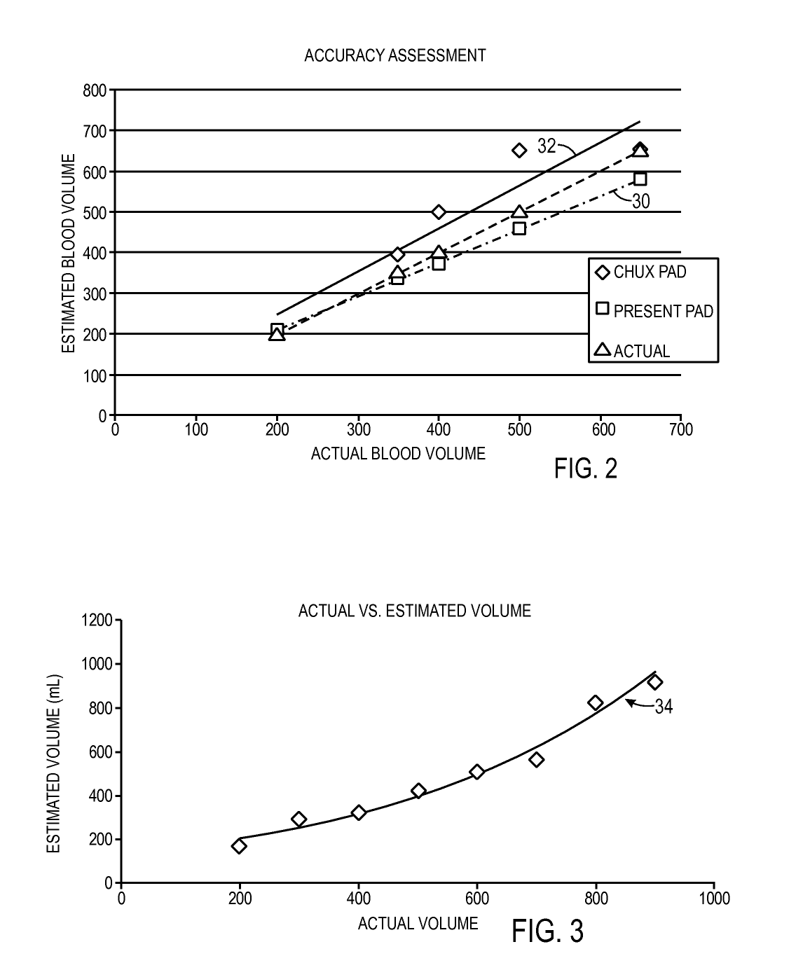

Based on this test, participant estimates made using the pad as disclosed herein underestimated blood loss by an average of 29.8 mL and were within 100 mL of the actual blood loss volume 78.8% of the time. Conversely, participant estimates made using the Chux pads overestimated blood loss by an average of 60.6 mL and were within 100 mL of the actual blood loss volume 75.1% of the time. Further, estimates made using the pad as disclosed herein followed a more consistent pattern than Chux pad estimates, suggesting that calibration would further increase the accuracy of the present pad. In addition, the pad as disclosed herein was found to be significantly more accurate than the Chux pad (p=0.07). Lastly, participants rated the pad as disclosed herein significantly easier to use than the Chux pad (p<0.001), Test results are summarized in Tables 1 and 2 below, as well as in FIG. 2. As depicted graphically in FIG. 2, the test results related to accuracy assessment of the pad as discussed herein may be characterized by the regression line 30 where y=0.84x+39.46, having an r²=0.99 while the test results related to the accuracy assessment of the Chux pads may be characterized by the regression line 32 where y=1.06x+37.51, having an r²=0.89.

TABLE 1
Estimates within 100 mL of Actual Blood Volume, N = 49

| Actual Blood Volume | Present Pad | Chux Pad |
|---|---|---|
| 200 mL | 97.9% | 89.8% |
| 350 mL | 75.5% | 77.6% |
| 400 mL | 79.6% | 67.3% |
| 500 mL | 65.3% | 57.1% |
| 650 mL | 75.5% | 83.6% |
| Average | 78.8% | 75.1% |

TABLE 2
Reported Ease of Use, N = 49

| | Present Pad | Chux Pad |
|---|---|---|
| Very Difficult | 4.1% | 34.7% |
| Difficult | 16.3% | 40.8% |
| Moderate | 48.9% | 22.4% |
| Easy | 26.5% | 0% |
| Very Easy | 2.0% | 0% |
| Average | Moderate | Difficult |

In a subsequent test, the accuracy of the pad as disclosed herein for estimating blood loss was assessed in person by participants, rather than via the intermediary of photographs. For the purpose of this test, accuracy was defined as being within 100 mL of the actual volume, which was deemed to be an acceptable range of error for clinical diagnosis.

In this test, participants viewed pads as disclosed herein in a laboratory setting. The pads had absorbed predetermined blood volumes in the range of 200 mL to 900 mL, in increments of 100 mL. After receiving instructions, participants estimated the volume of blood on each pad and recorded estimations on a data collection sheet. Participants included undergraduate students, graduate students, and university faculty. Based on this test regime, 82.3% of estimates of blood “loss” using the pad as discussed herein, were within 100 mL of actual blood sample volumes. Results of this test are presented in Table 3 and FIG. 3. As shown in FIG. 3, the fit 34 of the test results may be characterized by y=133.45 e^{0.022}x where r²=0.7.

TABLE 3
Blood Loss Estimations, N = 38

| Actual Blood Volume | Mean Pad Estimates | Estimates within 100 mL |
|---|---|---|
| 200 mL | 177 mL | 100% |
| 300 mL | 297 mL | 100% |
| 400 mL | 327 mL | 92.1% |
| 500 mL | 426 mL | 86.8% |
| 600 mL | 509 mL | 73.7% |
| 700 mL | 568 mL | 28.9% |
| 800 mL | 824 mL | 84.2% |
| 900 mL | 916 mL | 86.8% |
| Total | | 81.6% |

With the foregoing examples and results in mind, the above-described pad 10 and process will allow health-care workers in both the developing and the developed world to measure blood loss, identify postpartum hemorrhage, and subsequently manage the condition. The disclosed system provides an inexpensive approach for measuring blood loss during delivery or during other medical procedures. Health care providers in the developing world lack access to blood measurement devices due to cost and power constraints, and even hospitals in the developed world do not consistently measure blood loss due to the expense of currently available diagnostic tests. Thus the pad disclosed herein would offer accuracy in measurement and cost savings in hospitals in all corners of the world.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art, including combinations of aspects or features of the embodiments and examples disclosed herein. Such other examples are intended to be within the scope of the claims if they have structural elements that do not differ from the literal language of the claims, or if they include equivalent structural elements with insubstantial differences from the literal languages of the claims. The specific embodiments described above have been shown by way of example, and it should be understood that these embodiments may be susceptible to various modifications and alternative forms, including combinations of various features and aspects of the examples or embodiments discussed herein. It should be further understood that the claims are not intended to be limited to the particular forms disclosed, but rather to cover all modifications, equivalents, and alternatives falling within the spirit and scope of this disclosure.

1. A pad, comprising:
a backing layer; and
a grid of uniformly sized blood-absorbent structures attached to the backing layer.

2. The pad of claim 1, wherein each blood-absorbent structure is a three-inch square.

3. The pad of claim 1, wherein the grid is a five-by-five grid.

4. The pad of claim 1, wherein each blood-absorbent structure comprises:
two or more layers of fabric; and
an absorbent polymer.

5. The pad of claim 4, wherein the absorbent polymer comprises sodium polyacrylate.

6. The pad of claim 1, wherein the backing layer comprises:
a plastic layer; and
a matted cotton layer.

7. The pad of claim 1, where each blood-absorbent structure absorbs approximately 50 mL of blood.

8. The pad of claim 1, wherein the pad is capable of measuring blood loss volume with a 100 mL resolution.

9. A method for forming a blood-loss measurement pad, comprising:
forming fabric pieces of a given shape and size;
attaching two or more fabric pieces together to form a plurality of open fabric-layer structures;
placing a fixed amount of absorbent polymer into each open fabric layer structure;
closing an open side of each open fabric-layer structure to form a plurality of closed fabric-layer structures; and
attaching the plurality of closed fabric-layer structures to a backing layer in a grid-pattern.

10. The method of claim 9, wherein forming fabric pieces of a given shape and size comprises cutting fabric pieces into 3-inch squares.

11. The method of claim 9, wherein attaching two or more fabric pieces together comprises sewing or gluing the two or more fabric pieces together.

12. The method of claim 9, wherein the absorbent polymer comprises sodium polyacrylate.

13. The method of claim 9, wherein closing the open side of each open fabric-layer structure comprises sewing or gluing the respective open sides.

14. The method of claim 9, wherein the grid-pattern comprises a five-by-five grid.

15. The method of claim 9, wherein the backing layer comprises a plastic layer and a matted cotton layer.

16. A method for assessing blood loss, comprising:
placing a pad under a patient, wherein the pad comprises a plurality of blood absorbent structures;
counting a number of blood absorbent structures that are saturated with blood; and
estimating the blood loss volume of the patient based on the number of blood absorbent structures that are saturated with blood.

17. The method of claim 16, wherein each blood absorbent structure absorbs up to about 50 mL of blood.

18. The method of claim 16, wherein estimating the blood loss volume comprises multiplying the number of blood absorbent structures that are saturated with blood by a known blood-absorbance capacity for each blood absorbent structure.

19. The method of claim 16, comprising counting an additional number of blood absorbent structures that have absorbed blood but are not saturated with blood.

20. The method of claim 19, wherein estimating the blood loss volume of the patient is based on both the number of blood absorbent structures that are saturated with blood and the additional number of blood absorbent structures that have absorbed blood but are not saturated with blood.
